(19) United States
(12) Patent Application Publication
(10) Pub. No.: US 2008/0206211 A1
(43) Pub. Date: Aug. 28, 2008

(54) COMPOSITION CONTAINING A CELL CULTURE MEDIUM

(75) Inventor: Audrey GUENICHE, Rueil Malmaison (FR)

Correspondence Address: OBLON, SPIVAK, MCCLELLAND MAIER & NEUSTADT, P.C., 1940 DUKE STREET, ALEXANDRIA, VA, 22314, US

(73) Assignee: L'OREAL, Paris (FR)

(21) Appl. No.: 12/037,179

(22) Filed: Feb. 26, 2008

Related U.S. Application Data
(60) Provisional application No. 60/907,342, filed on Mar. 29, 2007.

(30) Foreign Application Priority Data
Feb. 26, 2007 (FR) 07 53495

(51) Int. Cl. A61K 35/74; C12N 1/20; A61Q 19/00; C12P 1/04

(52) U.S. Cl. 424/93.42; 435/253.4; 435/253.5; 435/252.9; 435/252.5; 424/93.44; 424/93.45; 424/93.46; 435/170

(57) ABSTRACT

Composition containing a conditioned cell culture medium or an extract thereof, said medium being able to be obtained by contact with at least one culture of digestive tract cells and at least one probiotic microorganism. It also relates to the use of conditioned culture medium for improving skin homeostasis, etc.

### REFERENCE TO PRIOR APPLICATIONS

This application claims priority to U.S. provisional application 60/907,342 filed Mar. 29, 2007, and to French patent application 0753495 filed Feb. 26, 2007, both incorporated herein by reference.

### FIELD OF THE INVENTION

The present invention relates to conditioned culture media and to the use thereof, in particular for improving homeostasis of the skin or of the mucous membranes. The invention also relates to the use of a conditioned culture media in the cosmetics and/or dermatological field, and in the field of reconstructed skin, the preparation and therefore the properties of which can be improved with conditioned media, or extracts thereof, according to the invention.

Additional aspects and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Human skin has two compartments, namely a superficial compartment, the epidermis, and a deep compartment, the dermis.

The epidermis is composed mainly of three types of cells, which are the keratinocytes (predominant), the melanocytes and the Langerhans cells. Each of these cell types contributes by means of its intrinsic functions to the essential role played in the body by the skin, in particular the role of protecting the body against outside attacks. The dermis provides the epidermis with a solid support. It is also its nourishing element. It is mainly constituted of fibroblasts and an extracellular matrix, itself composed mainly of collagen, elastin and a ground substance. Leukocytes, mast cells and tissue macrophages are also found therein. Finally, blood vessels and nerve fibres traverse the dermis.

The skin constitutes a barrier against outside attacks, in particular: chemical, mechanical and infectious attacks, and in this respect, a certain number of defence reactions against environmental factors (climate, ultraviolet rays, tobacco, pollution, infections, etc.) and/or xenobiotic factors (such as, for example, certain medicaments) occur therein.

It is therefore important to preserve or re-establish its integrity and the equilibrium of its various functions, in particular an equilibrium between the cell renewal and differentiation processes, or an optimum degree of moisturization. The generic term “skin homeostasis” is intended to mean the maintenance of functioning and of a physiological equilibrium of normal skin.

It is thus desirable to have novel ways for maintaining this skin homeostasis, in particular for maintaining the barrier function, and/or combating the signs of dehydration or of ageing of the skin.

It is also desirable to have improved culture media that make it possible to prepare single-cell, multicell or monolayer models or models in the form of reconstructed tissue, and in particular from skin cells in order to prepare keratinocyte cultures, fibroblasts, keratinocyte/melanocyte cocultures or reconstructed skin.

These aims and others are achieved through the use of “cell” culture media, or of extracts thereof, also referred to as conditioned media.

Application WO 02/098365 (Advanced Tissue Science) proposes the use of conditioned culture media for cosmetic or pharmaceutical applications. The conditioned media are obtained by culturing human skin cells, in particular fibroblasts or keratinocytes. These cells are genetically modified to increase their production of growth factors or of antioxidants in the medium, which generally contains a soluble collagen.

US 2005/0249691 describes cosmetic or dermatological compositions comprising a culture medium for cells of the skin or horny layers, in combination with a gelled matrix; the compositions necessarily contain collagen, chitosans and glycosaminoglycans.

US 2006/0182701 also describes cosmetic or dermatological compositions comprising a skin cell culture medium. It is aimed at providing the skin cells, to which the compositions are applied, with a medium which will allow them to develop in a manner similar to that which is obtained in vitro.

### SUMMARY OF THE INVENTION

Unexpectedly, it has now been found, in the context of the present invention, that a culture medium conditioned with cells completely different from skin cells can be used to promote good condition of the skin and/or of its appendages.

For this reason, a subject of the present invention is a composition comprising a cell culture medium or an extract thereof, said medium being able to be obtained by contact with at least one culture of digestive tract cells and at least probiotic microorganisms.

### DETAILED DESCRIPTION OF THE PREFERRED EMBODIMENTS

Advantageously, the cell culture medium, also called conditioned medium according to the present invention, can be obtained by contact with peripheral blood cells, or cells derived from peripheral blood cells. Such cells are in particular leukocytes, but can also be selected from immortalized cell lines, derived from blood cell lines, for instance from monocytes; non limiting example of such cells are those sold by LGC Promochem under the following designations:
SC (ATCC: CRL-9855)
AML-193 (ATCC: CRL-9589)
THP-1 (ATCC: TIB-202).

Mixtures may be used. These peripheral blood cells or derivatives thereof, such as leukocytes may, for example, be in culture in the medium with the digestive tract cells.

Preferably, these leukocytes comprise predominantly lymphocytes, but may also comprise other peripheral blood cells such as monocytes, which are brought into contact with the medium.

According to one of the advantageous embodiments of the invention, the culture of digestive tract cells is a three-dimensional culture.

The digestive tract cells used for the implementation of the invention may be derived from various parts of the digestive tract, such as the oesophagus, the stomach or the intestine. Advantageously, cells derived from the intestinal epithelium are used; such intestinal epithelial cells are known to those skilled in the art. By way of non-limiting example, mention may be made of the human cells of intestinal origin known as CaCO-2, HT29 or T84.

The corresponding strains are deposited in the culture collections of the ATCC with the following Nos.:
Caco 2: ATCC No. HTB-37
HT29: ATCC No. HTB-38
T84: ATCC No. CCL-248

Mixtures may be used.

For the purpose of the present invention, the term “probiotic microorganism” is intended to mean a living microorganism which, when it is consumed in adequate amount, has a positive effect on the health of its host, “joint FAO/WHO Expert Consultation on Evaluation of Health and Nutritional Properties of Probiotic in Food Including Powder Milk with Live Lactic Acid Bacteria, 6 Oct. 2001”, and which can in particular improve the intestinal microbial balance. Mixtures may be used.

The one or more probiotic microorganisms may be introduced into the cell culture medium in the form of a suspension of live cells, the concentration of which will be adjusted by those skilled in the art according to the amount of the other constituents of the mixture. By way of indication, it is possible to use an inoculum comprising approximately 10⁴ to 10⁹ cfu/ml (cfu signifying “colony forming unit”, i.e. “unit capable of forming a colony”), preferably at least 10⁵ cfu/ml. The probiotic inoculum may conventionally be at a concentration of approximately 10⁶ to 10⁷ cfu.

The microorganisms suitable for the invention may be chosen in particular from ascomycetes such as Saccharomyces, Yarrowia, Kluyveromyces, Torulaspora, Schizosaccharomyces pombe, Debaromyces, Candida, Pichia, Aspergillus and Penicillium, and bacteria of the genus Bifidobacterium, Bacteroides, Fusobacterium, Melissococcus, Propionibacterium, Enterococcus, Lactococcus, Staphylococcus, Peptostrepococcus, Bacillus, Pediococcus, Micrococcus, Leuconostoc, Weissella, Aerococcus, Oenococcus or Lactobacillus, and mixtures thereof.

As ascomycetes that are most particularly suitable for the present invention, mention may in particular be made of Yarrowia lipolitica and Kluyveromyces lactis, along with Torulaspora, Schizosaccharomyces pombe, Candida and Pichia, or alternatively yeasts such as Saccharomyces cerevisiae or boulardii. Mixtures may be used.

As regards the probiotic microorganisms, it is the following bacterial and yeast genera that are generally used:
lactic acid bacteria: which produce lactic acid by fermentation of sugar. They are divided up into two groups according to their morphologies:
   Lactobacillus species: Lactobacillus acidophilus (LC1, NCFB 1748); amylovorus, casei (Shirota), rhamnosus (strain GG), brevis, crispatus, delbrueckii (subsp bulgaricus, lactis), fermentum, helveticus, gallinarum, gasseri johnsonii, paracasei, plantarum, reuteri, rhamnosus, salivarius), alimentarius, curvatus, casei subsp. casei, sake;
   Gocci: Enterococcus (faecalis, faecium), Lactococcus lactis (subspp lactis or cremoris), Leuconstoc mesenteroides subsp dextranicum, Pediococcus acidilactici, Sporolactobacillus inulinus, Streptococcus salvarious subsp. Thermophilus, Streptococcus thermophilus, Staphylococcus carnosus, Staphylococcus xylosus;
bifidobacteria or Bifidobacterium species: Bifidobacterium adolescentis, animalis, bifidum, breve, lactis, longum, infantis, pseudocatenulatum;
the other sporulated bacteria: Bacillus (cereus var toyo or subtilis), Bacillus coagulans, Bacillus licheniformis, Escherichia coli strain nissle, Propionibacterium freudenreichii.

Mixtures may be used.

Specific examples of probiotic microorganisms are Bifidobacterium adolescentis, Bifidobacterium animalis, Bifidobacterium bifidum, Bifidobacterium breve, Bifidobacterium lactis, Bifidobacterium longum, Bifidobacterium infantis, Bifidobacterium pseudocatenulatum, Lactobacillus acidophilus (LCl, NCFB 1748); Lactobacillus amylovorus, Lactobacillus casei (Shirota), Lactobacillus rhamnosus (strain GG), Lactobacillus brevis, Lactobacillus crispatus, Lactobacillus delbrueckii (subsp bulgaricus, lactis), Lactobacillus fermentum, Lactobacillus helveticus, Lactobacillus gallinarum, Lactobacillus gasseri, Lactobacillus johnsonii, Lactobacillus paracasei, Lactobacillus plantarum, Lactobacillus reuteri, Lactobacillus rhamnosus, Lactobacillus salivarius), Lactobacillus alimentarius, Lactobacillus curvatus, Lactobacillus casei subsp. casei, Lactobacillus sake Lactococcus lactis, Enterococcus (faecalis, faecium), Lactococcus lactis (subspp lactis or cremoris), Leuconstoc mesenteroides subsp dextranicum, Pediococcus acidilactici, Sporolactobacillus inulinus, Streptococcus salvarius subsp. Thermophilus, Streptococcus thermophilus, Staphylococcus carnosus, Staphylococcus xylosus, Saccharomyces (cerevisiae or else boulardii), Bacillus (cereus var toyo or subtilis), Bacillus coagulans, Bacillus licheniformis, Escherichia coli strain nissle and Propionibacterium freudenreichii, and mixtures thereof.

Advantageously, at least one probiotic microorganism is chosen from lactic acid bacteria, bifidobacteria and Saccharomyces yeasts. Mixtures may be used.

More particularly, they are probiotic microorganisms derived from the lactic acid bacteria group, such as especially Lactobacillus and/or Bifidobacterium, in particular Lactobacillus. By way of illustration of these lactic acid bacteria, mention may more particularly be made of Lactobacillus johnsonii, Lactobacillus reuteri, Lactobacillus rhamnosus, Lactobacillus paracasei, Lactobacillus casei or Bifidobacterium bifidum, Bifidobacterium breve, Bifidobacterium longum, Bifidobacterium animalis, Bifidobacterium lactis, Bifidobacterium infantis, Bifidobacterium adolescentis or Bifidobacterium pseudocatenulatum, and mixtures thereof.

The species that are most particularly suitable are Lactobacillus johnsonii, Lactobacillus paracasei, Bifidobacterium adolescentis, Bifidobacterium longum and Bifidobacterium lactis NCC 2818 (also denoted Bb12 ATCC 27536); mention may in particular be made of the following strains, deposited according to the treaty of Budapest with the culture collection of the Institut Pasteur (28 rue du Docteur Roux, F-75024 Paris cedex 15) of 30/06/92, 12/01/99, 15/04/99, 15/04/99 and 07/06/05: Lactobacillus johnsonii (CNCM I-1225), Lactobacillus paracasei (CNCM I-2116), Bifidobacterium adolescentis (CNCM I-2168) and Bifidobacterium longum (CNCM I-2170) and Bifidobacterium lactis (CNCM I-3446), and the genus Bifidobacterium longum (BB536). The Bifidobacterium lactis CNCM I-3446 strain can be obtained from Hansen (Chr. Hansen A/S, 10-12 Boege Alle, P.O. Box 407, DK-2970 Hoersholm, Denmark). Mixtures may be used.

The cell culture medium with which the cells are brought into contact will be any nutritive medium suitable for the survival and/or the culture of the various cell types used. It generally contains a source of carbon and of nitrogen, minerals, vitamins and/or trace elements, for instance amino acids, sugars, proteins and fatty acids.

The cells may be cultured outside their tissue of origin. For this they should be cultured in an environment close to their natural condition in the tissue. These cultures require essential factors in their culture medium. This environment should have a defined composition composed of minerals and of biomaterials, known as culture medium. These culture media are in general provided by specialist suppliers and have specific characteristics according to the cell types. The culture media generally also contain water, a source of carbon and of nitrogen, phosphates and sulphates, minerals, growth factors and vitamins in a suitable amount.

The cells cultured in this type of medium often require the addition of serum. This serum has a complex composition and provides the cells at least with hormones, adhesion factors and amino acids. This serum may, for example, be entirely or partly replaced with the addition of the conditioned media of the invention. In fact, these conditioned media may also be added, in addition, to the culture medium, as enrichment.

Those skilled in the art will readily determine the suitable media in view of this disclosure. In a non-limiting manner, mention may be made of RPMI medium, Dulbecco's Modified Eagle's Medium (DMEM), Minimum Essential Medium (MEM), M199, RPMI 1640 or Iscove's Modified Dulbecco's Medium (EDMEM), Ham's F-12, Ham's F-10, NCTC 109 and NCTC 135.

These media may be supplemented with any additive including those normally used in cell culture, such as, for example, and in a non-limiting manner, phospholipid precursors, nonessential amino acids, nucleic acids, vitamins, antibiotics, enzymatic cofactors, mineral salts, insulin, transferrin, triiodothyronine, ethanolamine, o-phosphorylethanolamine or growth factors such as nerve growth factor or neurotrophin-3.

The concentrations of the various additives used for supplementing the cell culture media may be readily determined and adjusted by those skilled in the art in view of this disclosure, in particular according to the type of cells to be cultured.

Other media are described in HAM and McKeehan, “Methods in Enzymology”, 58:44-93, 1979, or else in Bottenstein et al., “Methods in Enzymology”, 58:94-109, 1979, the content of which is incorporated herein by way of reference.

Moreover, it is also possible to use mixtures of various media, in particular of the abovementioned media, such as for example a mixture of DMEM/HAM F12. These media may be supplemented with specific growth factors, or for example with serum, but the latter component may also be absent.

According to one embodiment, no collagen is added to the culture medium.

This culture medium may be liquid, semi-liquid, gelled or solid, preferably at least partially liquid.

The expression “extract of the conditioned cell culture medium” is intended to mean in particular any fraction or subcompound of these conditioned media obtained by dialysis, fractionation, phase separation, filtration chromatography, affinity chromatography, precipitation, concentration, lyophilization, etc.

The conditioned media may be generated from media which may be devoid of serum and of animal product. This conditioned medium is preferably obtained after stimulation of the digestive tract cells in the presence of human leukocytes, with probiotics and in particular probiotics of the lactobacillus or bifidobacterium species.

Advantageously, the culture medium (or extracts thereof) used in the compositions of the invention is a stabilized medium, i.e. it has been subjected to a manipulation intended to preserve it in the state in which it existed at a selected given instant, generally at the end of its preparation process, while at the same time having conserved its intrinsic properties. In particular, this manipulation is intended to render said medium sterile, i.e. incapable of allowing the growth of microorganisms, while at the same time preserving the biological properties that it possesses. The stabilization of the culture medium may be obtained by any technique known to those skilled in the art, such as, for example, sterilizing filtration, autoclaving, ultra-high temperature (UHT technique), high-pressure sterilization, γ-radiation or freezing.

The conditioned culture media according to the invention may generally contain IL-10, which was not present among the different constituents of the starting medium. The amount of IL-10 may vary, according to the conditions of preparation of the conditioned culture medium but will correspond generally to a concentration greater or equal to 20 pg/ml of the medium recovered after contact with the different cellular types.

Unexpectedly, it has now been found that such conditioned media have advantageous properties for the skin and/or the integuments.

For this reason, a subject of the present invention is also the use of at least one conditioned cell culture medium that can be or is obtained by contact with at least one culture of digestive tract cells and at least probiotic microorganisms, as defined above, or can be or is obtained by contact with at least one culture of digestive tract cells and at least one probiotic microorganism, as defined above, or of an extract of said cell culture medium, as an agent for improving the homeostasis of the skin and/or of the integuments.

Advantageously, the conditioned medium will be obtained by contact with, in addition, peripheral blood monocellular cells or derivatives thereof, in particular leukocytes.

In the present description, unless otherwise specified, the term “skin” is intended to mean the entire covering of the human body, i.e. the skin, the mucous membranes and the scalp.

The term “integuments” is intended to mean the nails, the hair and the body hairs, such as the eyelashes.

In particular, the invention relates to the use of a culture medium, or of an extract thereof, as an agent for improving the barrier function of the skin or of the scalp.

According to another aspect of the invention, the culture medium, or extracts thereof, is of use as an agent for improving the moisturization of the skin or of the scalp.

The culture media will thus be of particular use for combating the signs of dry skin, in particular dry and sensitive skin.

In general, sensitive skin is defined by a particular reactivity of the skin.

This skin reactivity is generally reflected by the manifestation of signs of discomfort in response to the individual coming into contact with a triggering element that may have various origins. This may be the application of a cosmetic product at the surface of the sensitive skin, food intake, or exposure to abrupt variations in temperature, to atmospheric pollution and/or to ultraviolet or infrared rays. Associated factors such as age and skin type also exist. Thus, sensitive skin is more common in the case of dry or oily skin according to the cosmetic criteria for skin type classification. Individuals with sensitive skin may thus be individuals with irritable or reactive skin, or with intolerant skin.

The appearance of these signs of discomfort, which appear within minutes following the individual coming into contact with the triggering element, is one of the essential features of sensitive skin. It involves mainly dysaesthetic sensations. The term “dysaesthetic sensation” is intended to mean sensations felt in an area of the skin, such as stinging, tingling, itching, sensations of hotness, discomfort, tautness, etc. These subjective signs exist most commonly in the absence of visible chemical signs such as redness or desquamations, and in any event without there being any oedema. It is today known that these skin irritation and intolerance reactions are in particular related to a release of neuropeptides by the nerve endings of the epidermis and of the dermis.

During acute manifestations of skin sensitivity, a neurogenic reaction may be triggered. Its long-term consequences may contribute to maintaining the inflammatory or hyperalgesic phenomena, and therefore lead to the chronicity of the process.

Unlike skin described as allergic skin, the reactivity of sensitive skin is not the result of an immunological process, i.e. does not occur only in skin that has already been sensitized, in response to the presence of an allergen. Its response mechanism is terms “aspecific”. It is, in this respect, to be distinguished from skin manifesting inflammatory and allergic reactions of dermatosis, eczema and/or ichthyosis type.

In the case of the invention, the conditioned media allow the prevention and/or treatment of skin described as sensitive skin, in particular when this sensitive skin is associated with dry skin. Dry skin manifests itself essentially through a sensation of tautness and/or of tension. It is commonly associated with a decrease in the degree of moisturization of the skin and an impairment of the barrier function, measured through the insensible water loss.

As specified above, sensitive skin is different from allergic skin. Its reactivity is not the result of an immunological process and is generally reflected only by dysaesthetic sensations, possibly associated with redness, but without swelling or oedema.

“Sensitive skin” can in particular be characterized using tools for evaluating the sensory reaction of the skin. The first tools were based, right from their design, on the essential feature of dry skin, namely the presence of signs of discomfort induced by a topical application. Thus the lactic acid “stinging test” was the first test proposed. It is carried out by recording the stinging sensations reported by a volunteer after application of a 10% lactic acid solution to the wings of the nose. Individuals who report moderate or strong stinging sensations are called “stingers” and are considered to have sensitive skin. Because of this sensitivity of the skin to the topical application of a product, these individuals are then selected for testing “sensitive skin” products. More recently, in order to specifically activate the peripheral nerve endings involved in the discomfort and called nociceptors, recently identified as being involved in sensitive skin, new tests have been proposed that use precisely other inducers of discomfort such as capsaicin, in particular in application EP 1 374 913. For the purpose of the present invention, the term “sensitive skin” covers irritable skin and intolerant skin.

Intolerant skin is skin that reacts to various factors, such as the application of cosmetic or dermatological products or soap, through sensations of hotness, tautness, tingling and/or redness.

Irritable skin is skin that reacts through itching or through stinging, to various factors such as the environment, emotions, foods, wind, friction, shaving, hard water with a high calcium content, temperature variations, humidity or wool.

These two types of skin are generally associated with dryness of the skin with or without dry patches, or with skin that exhibits erythema. Intolerant skin may, however, also be associated with acneic skin, or even skin exhibiting rosacea, with or without dry patches.

As specified above, dryness of the skin is often associated with a decrease in the degree of moisturization of the skin, evaluated by corneometry, and with an impairment of the barrier function, measured through the insensible water loss.

Dry skin essentially manifests itself through a sensation of tautness and/or of tension. Said skin is also rough to the touch and appears to be covered with scales. When the skin is slightly dry, these scales are abundant but not very visible to the naked eye.

The cause of this dryness of the skin may be of the constitutional or acquired type.

In the case of acquired dry skin, the involvement of outside parameters such as exposure to chemical agents, to difficult climatic conditions or to sunlight, or alternatively certain therapeutic treatments (retinoids, for example) is determinant. Under these outside influences, the skin may then become momentarily and locally dry. This can involve any type of skin.

In the case of nonpathological constitutional dry skin, the severity of the state of dryness can depend on the outside factors already mentioned. Senile skin (characterized by a general decrease in skin metabolism with age), fragile skin (very sensitive to outside factors and often accompanied by erythema and rosacea) and common xerosis (of probable genetic origin and manifesting itself predominantly on the face, the limbs and the back of the hands) fall into this skin category. It should be understood that the dry skin state is a physiological state which is independent of any pathological manifestation and corresponds to a normal skin, the comfort and the appearance of which it is desired to improve by cosmetic means.

The compositions, methods and uses according to the invention thus prove to be most particularly effective for preventing and/or treating sensitive and/or dry skin, and more particularly skin referred to as reactive, irritable and/or intolerant, acquired dry skin and/or constitutional dry skin.

A subject of the invention is also the use of a conditioned medium or of an extract thereof, for combating the signs of ageing, whether chronological or photoinduced ageing. Over time, various signs very characteristic of intrinsic ageing appear on the skin, reflected in particular by a modification of the structure and of the functions of the skin. The other component is exogenous (Yaar and Gilchrest, J Invest Dermatol, 1998). In fact, the ageing may be accelerated by environmental factors such as repeated exposure of the skin to sunlight, and in particular to ultraviolet A and B radiation, to pollution, in particular to diesel particles, and to cigarette smoke.

The toxicity of atmospheric pollutants, in particular gas pollutants such as sulphur dioxide, ozone and nitrogen oxides, on the constituents of the skin (fibres, cells, enzymes) is related in particular to their initiating activity on free radicals, which are sources of oxidation phenomena that cause cell damage in living beings. This damage induces accelerated ageing of the skin.

The signs of skin ageing are in particular wrinkles and fine lines, flaccid skin or thinned skin.

One of the subjects of the invention is the use of a conditioned medium or an extract thereof, in or for the preparation of a composition, in particular of a cosmetic composition, the conditioned medium, the extract thereof and/or the composition being for use in improving skin homeostasis and/or the barrier function and/or the moisturization of the skin, of the scalp and/or of the integuments.

A subject of the invention is also a process for improving skin homeostasis and/or the barrier function and/or the moisturization of the skin or of the scalp, wherein at least one cell culture medium or at least one culture medium extract or a composition containing the same, is applied to the skin or the scalp, said medium being able to be obtained by contact with at least one culture of digestive tract cells and at least probiotic microorganisms, as defined in the above text or with at least one culture of digestive tract cells and at least one probiotic microorganism, as defined in the above text.

According to another of its aspects, the invention relates to the use of at least one cell culture medium or at least one culture medium extract, said medium being able to be obtained by contact with at least one culture of digestive tract cells and at least probiotic microorganisms, as defined in the above text, or obtained by contact with at least one culture of digestive tract cells and at least one probiotic microorganism, as defined in the above text, for the preparation of a composition for use in the treatment and/or prevention of the signs of skin stress. Such a composition may be a composition for cosmetic or pharmaceutical use, in particular a dermatological composition.

The amount of conditioned culture medium or of extracts thereof according to the invention in the compositions will be adjusted by those skilled in the art so as to obtain the desired effect. The effective amount will thus be determined by routine techniques, comprising in vitro tests and in vivo assays, and will depend in particular on the type of extract used and on the type of formulation selected.

By way of indication, and not limitation, the concentration of conditioned medium active material may be between 0.001% and 50% by weight relative to the total weight of the composition, in particular less than or equal to 10%, but these amounts may vary without any drawback.

The compositions according to the invention also contain a physiologically acceptable medium, in particular a cosmetically or pharmaceutically acceptable medium.

For the purpose of the invention, the term “cosmetic composition or product” is intended to mean in particular any substance or preparation intended to be brought into contact with the various superficial parts of the human body (epidermis, head hair and body hair system, nails, lips and external genital organs) or with the teeth and the oral mucous membranes, with a view, exclusively or mainly, to cleaning them, fragrancing them, modifying the appearance thereof and/or correcting body odours and/or protecting them or maintaining them in good condition (Cosmetic Guideline 76/768/EEC amended).

These compositions generally have an odour and an appearance that makes them pleasant to apply to the human body.

Preferably, a composition of the invention is applied to the skin or the mucous membranes.

According to the method of administration under consideration, it may be in any of the galenical forms normally used.

For topical application to the skin or the mucous membranes, the composition may be in the form in particular of aqueous or oily solutions or dispersions of the lotion or serum type, emulsions with a liquid or semi-liquid consistency of the milk type, obtained by dispersion of a fatty phase in an aqueous phase (O/W) or vice versa (W/O), or suspensions or emulsions with a soft consistency of the aqueous or anhydrous cream or gel type, or else microcapsules or microparticles, or vesicular dispersions of ionic and/or non-ionic type, or foams. They may also be in the form of microspheres or nanospheres or of lipid or polymeric vesicles or of polymeric patches and of hydrogels for controlled release.

According to an advantageous embodiment, the composition is a dermocosmetic composition containing, in a cosmetically or pharmaceutically acceptable support, at least one conditioned medium or one extract according to the invention, in a proportion of at least 0.001% by weight relative to the total weight of the composition, and preferably from 0.05% to 3%.

These compositions are prepared according to the usual methods.

The amounts of the various constituents of the compositions according to the invention are those normally used in the fields under consideration. The constituents and the amounts thereof will preferably be chosen so as not to interact with the activity of the conditioned medium, or of extracts thereof, by decreasing said activity.

In the cosmetics field, these compositions constitute in particular cleansing, protection, treatment or care creams for the face, for the hands, for the feet, for the large anatomical folds or for the body (for example, day creams, night creams, makeup-removing creams, foundation creams, anti-sun creams), fluid foundations, makeup-removing milks, body protection or care milks, anti-sun milks, skincare lotions, gels or foams, such as cleansing lotions, artificial-tanning lotions, bath compositions, deodorant compositions comprising a bactericidal agent, or aftershave gels or lotions.

The compositions according to the invention may also consist of solid preparations constituting soaps or cleansing bars.

The compositions may also be packaged in the form of an aerosol composition, also comprising a pressurized propellant.

A composition according to the invention may also be a scalp care composition, in particular a shampoo, a hairsetting lotion, a treating lotion, a styling gel or cream, restructing lotions for the hair, a lotion or a gel for combating hair loss, an antiparasitic shampoo, antidandruff shampoo, etc.

A composition may also be for orodental use, for example a toothpaste. In this case, the composition may contain normal adjuvants and additives for compositions for oral use, and in particular surfactants, thickeners, humectants, polishing agents such as silica, various active ingredients such as fluorides, in particular sodium fluoride, and optionally sweeteners such as sodium saccharinate.

When the composition is an emulsion, the proportion of the fatty phase may vary from approximately 5% to 80% by weight, and preferably from approximately 5% to 50% by weight, relative to the total weight of the composition. The oils, the waxes, the emulsifiers and the coemulsifiers used in the composition in emulsion form are chosen from those conventionally used in the cosmetics field. The emulsifier and the coemulsifier are present, in the composition, in a proportion ranging from 0.3% to 30% by weight, and preferably from 0.5% to 20% by weight, relative to the total weight of the composition. The emulsion may also contain lipid vesicles.

When the composition is an oily solution or gel, the fatty phase may represent more than 90% of the total weight of the composition.

In a known manner, the cosmetic composition may also contain adjuvants common in the cosmetics field, such as hydrophilic or lipophilic gelling agents, hydrophilic or lipophilic additives, preserving agents, antioxidants, solvents, fragrances, fillers, screens, odour absorbers and dyestuffs. The amounts of these various adjuvants are those conventionally used in the cosmetics field, and, for example, range from approximately 0.01% to 10% of the total weight of the composition. Depending on their nature, these adjuvants may be introduced into the fatty phase, into the aqueous phase and/or into the lipid spherules.

As oils or waxes that can be used in the invention, mention may be made of mineral oils (liquid petroleum jelly), vegetable oils (liquid fraction of shea butter, sunflower oil), animal oils (perhydrosqualene), synthetic oils (purcellin oil), silicone oils or waxes (cyclomethicone) and fluoro oils (perfluoropolyethers), beeswax, carnauba wax or paraffin wax. Fatty alcohols and fatty acids (stearic acid) may be added to these oils. As emulsifiers that can be used in the invention, mention may, for example, be made of glyceryl stearate, polysorbate 60 and the mixture of PEG-6/PEG-32/glycol stearate sold under the name Tefose® 63 by the company Gattefosse.

As solvents that can be used in the invention, mention may be made of lower alcohols, in particular ethanol and isopropanol, and propylene glycol.

As hydrophilic gelling agents that can be used in the invention, mention may be made of carboxyvinylpolymers (Carbomer®), acrylic copolymers such as acrylate/alkyl acrylate copolymers, polyacrylamides, polysaccharides such as hydroxypropylcellulose, natural gums and clays, and, as lipophilic gelling agents, mention may be made of modified clays such as bentones, metal salts of fatty acids such as aluminium stearates, hydrophobic silica, ethylcellulose and polyethylene.

In the dermocosmetic compositions according to the invention, the conditioned medium extract may be combined with retinoids or corticosteroids, or associated with free-radical scavengers, with alpha-hydroxy or alpha-keto acids or derivatives thereof, or alternatively ion channel blockers.

The dermocosmetic compositions according to the invention may also contain inert or even pharmacodynamically or cosmetically active additives or combinations of these additives, and in particular: wetting agents, depigmenting agents such as hydroquinone, azelaic acid, caffeic acid or kojic acid; emollients; moisturizing agents such as glycerol, PEG-400, urea; anti-ageing agents, anti-seborrhoeic or anti-acne agents, such as S-carboxymethylcysteine, S-benzylcysteamine, salts thereof and derivatives thereof, or benzoyl peroxide; antibiotics such as erythromycin and esters thereof, neomycin, clindamycin and esters thereof, tetracyclines; antifungal agents such as ketoconazole or 4,5-polymethylene-3-isothiazolinones; agents for promoting hair regrowth, such as Minoxidil (2,4-diamino-6-piperidinopyrimidine 3-oxide) and derivatives thereof, Diazoxide (7-chloro-3-methyl-1,2,4-benzothiadiazine 1,1-dioxide) and Phenytoin (5,4-diphenylimidazoline-2,4-dione); nonsteroidal anti-inflammatory agents, carotenoids and in particular β-carotene; antipsoriatic agents such as anthralin and derivatives thereof, and, finally, eicosa-5,8,11,14-tetraenoic acid and eicosa-5,8,11-trienoic acid, esters thereof and the amides. The anti-ageing agents are in particular antiglycation agents, NO-synthase inhibitors, or agents for stimulating the synthesis of dermal or epidermal macromolecules or preventing their degradation, or agents for stimulating fibroblast or keratinocyte proliferation and/or keratinocyte differentiation. The term “moisturizing agent” is intended to mean:

either a compound that acts on the barrier function, in order to maintain the moisturization of the stratum corneum, or an occlusive compound. Mention may be made of ceramides, sphingoid-based compounds, lecithins, glycosphingolipids, phospholipids, cholesterol and derivatives thereof, phytosterols (stigmasterol, β-sitosterol, campesterol), essential fatty acids, 1,2-diacylglycerol, 4-chromanone, pentacyclic tri-terpenes such as ursolic acid, petroleum jelly and lanolin;

or a compound that directly increases the water content of the stratum corneum, such as trehalose and derivatives thereof, hyaluronic acid and derivatives thereof, glycerol, pentanediol, sodium pidolate, serine, xylitol, sodium lactate, polyglyceryl acrylate, ectoin and derivatives thereof, chitosan, oligosaccharides and polysaccharides, cyclic carbonates, N-lauroylpyrrolidonecarboxylic acid and N-α-benzoyl-L-arginine;

or a compound that activates the sebaceous glands, such as steroid derivatives (including DHEA), and vitamin D and derivatives thereof.

These compounds may represent from 0.001% to 30%, and preferably from 0.01% to 20% of the total weight of the composition according to the invention.

Examples of NO-synthase inhibitors that are suitable for use in the present invention include, in particular, an extract of a plant of the species Vitis vinifera which is in particular sold by the company Euromed under the name Leucocyanidines de raisins extra, or by the company Indena under the name Leucoselect®, or, finally, by the company Hansen under the name Extrait de marc de raisin; an extract of a plant of the species Olea europaea which is preferably obtained from olive tree leaves and is in particular sold by the company VINYALS in the form of a dry extract, or by the company Biologia & Technologia under the trade name Eurol BT; and an extract of a plant of the species Gingko biloba which is preferably a dry aqueous extract of this plant sold by the company Beaufour under the trade name Ginkgo biloba extrait standard.

Among the active agents for stimulating dermal macromolecules or preventing their degradation, mention may be made of those which act:

either on collagen synthesis, such as extracts of Centella asiatica; asiaticosides and derivatives; ascorbic acid or vitamin C and its derivatives, such as ascorbyl glucoside (sold by the company Hayashibara); synthetic peptides such as iamin, the palmitoyl tripeptide glycine-histidine-lysine sold under the name “Biopeptide CL” by the company Sederma; peptides extracted from plants, such as the soybean hydrolysate sold by the company Coletica under the trade name Phytokine®; soybean fibre extracts, such as that sold under the name “Raffermine” by the company Silab; plant hormones such as auxins and lignans; the palmitoyl pentapeptide lysine-threonine-threonine-lysine-serine sold in particular under the name “Matrixyl” by the company Sederma; dimethylaminoethanol; extracts of Bupleurum chinensis rhizome, such as those sold under the names “Pleurimincyl” and “Lipocare” by the company Sederma; wheat protein hydrolysates acylated in particular with a palmitoyl group, such as that sold under the name “Lipacid PVB” by the company Seppic; creatine; coenzyme Q10; retinol, dipalmitoyl hydroxyproline, in particular sold by the company Seppic under the name “Sepilift DPHP”, and extracts of red clover (Trifolium pratense) containing isoflavones;

or on elastin synthesis, such as the extract of Saccharomyces cerivisiae sold by the company LSN under the trade name Cytovitin®; and the extract of the alga Macrocystis pyrifera sold by the company Secma under the trade name Kelpadelie®;

or on glycosaminoglycan synthesis, such as the product of fermentation of milk by lactobacillus vulgaris, sold by the company Brooks under the trade name Biomin yogourth®; the extract of the brown alga Padina pavonica sold by the company Alban Muller under the trade name HSP3®; and the extract of Saccharomyces cerevisiae available in particular from the company Silab under the trade name Firmalift® or from the company LSN under the trade name Cytovitin®;

or on fibronectin synthesis, such as the extract of the zooplancton Salina sold by the company Seporga under the trade name GP4G®; the yeast extract available in particular from the company Alban Muller under the trade name Drieline®; and the palmitoyl pentapeptide sold by the company Sederma under the trade name Matrixil®;

or on the synthesis of compounds present at the level of the dermal-epidermal junction (such as collagens VII and/or IV and/or laminin), such as dipalmitoyl hydroxyproline, in particular sold by the company Seppic under the name “Seppilift DPHP”, or phytosteryl sulphate, such as that sold by the company Vincience under the name “Phytocohesine”;

or on the inhibition of metalloproteinases (matrix metalloproteinases or MMPs), such as more particularly MMP 1, 2, 3 or 9. Mention may be made of: retinoids and derivatives, oligopeptides and lipopeptides, lipoamino acids, the malt extract sold by the company Coletica under the trade name Collalift®; extracts of blueberry or of rosemary; lycopene; isoflavones, their derivatives or the plant extracts containing them, in particular the extracts of soybean (sold, for example, by the company Ichimaru Pharcos under the trade name Flavosterone SB®), of red clover (sold, for example, by the company Sederma under the name “Sterocare®”), of flax, of kakkon or of sage; extracts of Cucurma longa; extracts of Siegesbeckia (sold, for example, by the company Sederma);

or on the inhibition of serine proteases such as leukocyte elastase or cathepsin G. Mention may be made of: the peptide extract of legume seeds (Pisum sativum) sold by the company LSN under the trade name Parelastyl®; heparinoids; and pseudodipeptides such as {2-[acetyl-(3-trifluoromethylphenyl)amino]-3-methyl-butyrylamino}acetic acid.

The composition according to the invention may also contain preserving agents, such as para-hydroxybenzoic acid esters, stabilizers, humidity regulators, emulsifiers, UVA and UVB screens, and antioxidants, such as alpha-tocopherol, butylhydroxyanisole or butylhydroxytoluene.

According to another aspect of the invention, it relates to the use of at least one cell culture medium or of at least one culture medium extract, said medium being able to be obtained by contact with at least one culture of digestive tract cells and at least probiotic microorganisms, as defined above, or obtained by contact with at least one culture of digestive tract cells and at least one probiotic microorganism, as defined in the above text, for the preparation of a cell and/or tissue culture medium. The conditioned culture medium according to the invention, or extracts thereof, may thus be used as an adjuvant in cell culture systems, in particular for improving three-dimensional systems and obtaining optimal conditions for culturing in submerged or emerged systems. In particular, the conditioned culture medium and/or extracts thereof (and/or compositions containing them) may be used for the preparation of an in vitro skin equivalent.

In fact, the conditioned cell culture medium or extracts thereof may advantageously be used alone or as a mixture with a conventional culture medium, for culturing cells in vitro, in particular skin cells such as keratinocytes.

Such culture systems have in particular been described, for example, in patents EP285471, EP789074 or EP857971, and comprise a three-dimensional culture of more or less differentiated keratinocytes on a support. The culture may also contain other skin cell types, such as melanocytes, dendritic cells or Langerhans cells, fibroblasts, but also nerve cells or endothelial cells.

The support may be constituted of an inert support, in particular a porous or semi-permeable support. The support may more particularly be chosen from a collagen matrix, optionally comprising fibroblasts and/or glycosaminoglycans, a de-epidermalized dermis, a dermis equivalent, a hyaluronic acid and/or collagen and/or fibronectin and/or fibrin membrane, and an inert support.

A subject of the invention is also a method for preparing a composition, in particular a cosmetic or dermatological composition, comprising a first phase during which a conditioned culture medium, optionally an extract of such a conditioned culture medium, is prepared.

This method comprises at least the following steps:
a) culturing cells derived from the intestinal epithelium on a support, in particular a porous support, in a first nutritive medium for a period of time sufficient to obtain their differentiation;
b) preparing a second cell culture medium in a chamber covered with a carpet of peripheral blood cells or derivatives thereof, such as leukocytes;
c) recovering the culture of differentiated cells on the support, in particular porous support, obtained at the end of step a), and transferring to the second culture medium obtained at the end of step b);
d) bringing said culture of differentiated cells derived from the intestinal epithelium in the second culture medium into contact with a culture of microorganisms comprising at least probiotics which may be, for example, of the Lactobacillus and/or Bifidobacterium species, for a period of time sufficient for there to be an interaction between the cells;
e) eliminating the cell cultures and recovering the second cell culture medium, freed of the leukocytes, so as to obtain a conditioned medium;
f) incorporating the conditioned medium or an extract thereof into a cosmetic or dermatological composition.

Preferably, the support used in step a) and seq. is a porous support.

The term “porous support” is intended to mean in particular an insert whose base comprises pores.

For example, the pore size may range from 0.001 to 10 μm, preferably greater than or equal to 0.3 μm.

By way of non-limiting example, the base of the porous insert that is suitable for the invention may thus comprise a porous collagen matrix, optionally comprising glycosaminoglycans and/or fibroblasts, a hyaluronic acid and/or collagen and/or fibronectin and/or fibrin gel or membrane, a semi-permeable nitrocellulose, nylon, teflon, polycarbonate, polyethylene, polypropylene or polyethylene terephthalate (PET) membrane, a semi-permeable Anopore® inorganic membrane, a cellulose acetate membrane, a Biopore-CM® semi-permeable membrane, a semi-permeable polyester membrane and a polyglycolic acid membrane.

The contacting time in step a) would be adjusted by those skilled in the art, but will generally be between a few hours and a few days, in particular between 1 day and 35 days, for example from 18 to 22 days, preferably approximately 21 days. The cells which are initially arranged in a single layer thus form, at the end of step a), a three-dimensional multilayer system of differentiated cells. Advantageously, the culture medium will be renewed regularly, for example every two days, so as to obtain optimal differentiation of the cells derived from the intestinal epithelium, such as Caco2 cells.

In step b), the peripheral blood cells or derivatives thereof, in particular the leukocytes form a carpet of cells; i.e. the surface of the chamber is substantially homogeneously covered with leukocytes; they may be in the form of a monolayer or in multilayers.

These leukocytes will in particular be collected from a blood sample after at least one separation step, in particular by centrifugation, and then at least partial elimination of the monocytes. They are subsequently resuspended in a nutritive medium compatible with their viability, in particular RPMI medium.

In general, the media may be chosen by those skilled in the art according to their general knowledge, and in particular from the media mentioned above.

By way of example of a chamber suitable for implementing the invention, mention may be made of the wells of culture plates such as 6-, 12-, 24-, 48-well or 96-well cell culture plates, normally used in cell culture.

Advantageously, the culture of differentiated cells will be washed with the medium suitable for leukocyte viability before the transfer in step c). The contacting between the differentiated cells and the probiotics, in the presence of the leukocytes, will be carried out for a period of time that allows the establishment of interaction, i.e. chemical or biological exchanges, between the various cell types.

For the purpose of the invention, the expression “cellular chemical exchange” is intended to denote all the signals represented by molecules, released from a cell and capable of affecting, remotely, the activity of another cell, which may or may not belong to the same cell type. Such a molecule may, for example, and in a non-limiting manner, be a peptide, a protein, a lipid, a sugar, a steroid hormone or a catecholamine. It may be released in the form of a secretion.

It is understood that this interaction may occur in the absence of direct physical contact between the various cell types; in particular, the arrangement by which, firstly, the cells derived from the intestinal epithelium and, secondly, the carpet of leukocytes, which may or may not be placed in a single chamber, are brought into communication with one another by means of the culture medium in which they are incubated, without cells of the culture of intestinal cells being able to come directly into contact with cells of another carpet of cells, for example by contact between the cell bodies or by means of cell extensions.

Advantageously, the probiotic microorganisms are added to the intestinal cell culture apically.

The period of time suitable for the establishment of this interaction will be from a few hours to several days, generally at least 6 hours, preferably at least 10, in particular greater than or equal to 16 hours, but may be extended without any disadvantage. The probiotics are, for example, left in contact with the intestinal cells in the chamber comprising the leukocytes for 6 to 36 hours.

The culture medium is then recovered by separating it from all the cells, for example by harvesting it basolaterally: this conditioned medium has been influenced by the two cell types and their interrelationship.

As indicated before, the culture medium such recovered, also called “conditioned medium” contains IL-10 in a concentration greater or equal to 20 pg/ml, in particular from 50 to 200 pg/ml.

It may then be subjected to concentration, extraction and/or fractionation steps known to those skilled in the art, before the introduction, as ingredient, into a composition, in particular a cosmetic, pharmaceutical or dermocosmetic composition according to the invention.

The composition according to the invention is for use in particular in the therapeutic or prophylactic treatment of normal skin and/or mucous membranes and/or affected skin and/or mucous membranes that may exhibit disorders of the skin, the integuments and/or the hair, such as, in particular:

dry skin or hair,

impairment of the skin barrier function,

sensitive skin or hair,

skin with alterations in its extracellular matrix,

disorders related to therapeutics with antibiotics or antimycotic agents,

disorders caused by hormone disturbances (greasy skin) or related to dandruff.

Furthermore, the composition may prevent or treat certain impairments related to chronological ageing.

The invention will be illustrated in greater detail in the following examples.

In these examples, reference will be made to the attached figure, which shows the conditions for penetration of a reference compound into reconstructed skin cultured under various conditions.

### EXAMPLE 1

### Preparation of a Conditioned medium

Cells close to human enterocytes, Caco2 (between passage 60 and 65) are seeded at the density of 2.5×10⁵ cells/ml in a 25 mm culture insert (having nucleopores of 0.4 μm, Becton Dickinson, Basle, Switzerland). These inserts are placed in a culture dish (Nunc) and cultured for 18 to 22 days at 37° C./10% CO₂ in DMEM (containing glutamine and a high concentration of glucose (Amimed, Allschwill, Switzerland)) supplemented with nonessential amino acids (Gibco, BRL), 10 mg/ml gentamycin (Gibco BRL) and 0.1% of penicillin/streptomycin (10 000 IU/ml and 10 000 μg/ml) (Gibco). The culture medium is changed every 2 days until the cells are completely differentiated (21 days). A measurement of the transepithelial electrical resistance is determined continually when the Caco2 cells are confluent in a monolayer, using a Multicell-ERS electrode (voltmeter/ohmmeter).

Moreover, blood leukocytes of normal volunteers are isolated from peripheral blood from unrelated normal donors, by centrifugation on Lymphoprep. The cell suspensions (10⁷ cell/ml) are placed in a Petri dish and incubation for 1 h 30 at 37° C. allows the monocytes to adhere. Thus, the leukocytes, predominantly T lymphocytes, contained in the population of non-adherent cells are purified using their property of forming rosettes in the presence of sheep red blood cells. The latter are then eliminated by osmotic shock in the presence of NH₄Cl (8.7 mg/l). In all cases, the viability of the leukocyte suspension is greater than 95%.

These leukocytes are then diluted in RPMI 1640 containing 20% of decomplemented human AB serum (decomplemented at 56° C. for 30 minutes, Sigma, St Louis, Mo., USA).

The Caco2/leukocyte coculture model can then be prepared. For this, the Caco2 cell culture inserts are washed twice in RPMI 1640 medium and transferred into a 6-well culture plate containing RPMI medium, covered beforehand with a carpet of freshly purified leukocytes (2×10⁶ cell/ml). Thus, the leukocytes are at the basolateral level and the Caco2 cells are at the apical level.

The stimulation of the Caco2/leukocyte (peripheral blood white blood cells) cocultures with probiotics is carried out according to the conditions described below:

Thus, 1×10⁷ cfu/ml of probiotics are added apically. The culture medium alone is used as negative control. After stimulation of the coculture for 6 to 36 h (37° C., 10% CO2), the conditioned medium is harvested basolaterally.

Lactobacillus paracasei, especially the strain deposited at the CNCM under No. CNCM I-2116, is in particular used as probiotic.

### EXAMPLE 2

### Effect on the Barrier Function

Conditioned media obtained according to Example 1 but after 16 h of stimulation with 10⁸ cfu of Lactobacillus paracasei were brought into contact with Episkin reconstructed skin for 6 days during the phase of this model which was still proliferative. The skin barrier function of this resulting Episkin® model was then studied.

### Protocol

The Episkin kits were received at D6, and then cultured during the proliferative phase until D13 according to five conditions:
1. Conventional Episkin condition (condition A):

treated from D6 to D13 with the Episkin® differentiation medium.

The Episkin differentiation medium is that described by Roguet R, Cohen C, Dossou KG, Rougier A: Episkin, a reconstructed human epidermis for assessing in vitro the irritancy of topically applied compounds (Toxicol in vitro 1993: 8: 283-291).
2. Negative control (condition B): treated from D6 to D13 with 30% of negative-control culture medium (conditioned medium derived from 16 h 00 of Caco2/PBMC culture).
3. Positive control (condition C): treated from D6 to D13 with 30% of conditioned medium Caco2/PBMC+probiotic (Lactobacillus paracasei CNCM I-2116+Bifidobacterium lactis NCC 2818 (also denoted Bb12 ATCC27536)).

Thus, the kits were received at D14 on culture medium. The culture medium was then replaced with test medium (which is equivalent in terms of its constitution to the differentiation medium, with, in place of 10% of foetal calf serum (FCS), only 5% of FCS) for 24 hours in an incubator at 37° C.

This test medium was then replaced with 1.5 ml of PBS+0.25% (w/w) tween 80 and then stabilized at 32° C. for 1 hour before application. The Episkin kits were used in their unit.

Conditions A, B, C were studied on 6 wells for each Episkin® batch.

The penetration of the compound 2-nitro-para-phenylene-diamine (dye) is studied after various treatments.

Experimental Conditions

The test is carried out as follows:
i) Formulation: mixture between the formulation containing the dye at 5 mM (1 part) and of a buffer solution, pH 9.5 (1 part). Final concentration of the dye in the formulation: 1 mM of starting material.

| | | Supplier | Per 100 g | |
|---|---|---|---|---|
| Formulation 1 | Alkyl (C8/C10 | SEPIC: oramix | 12 | g |
| | 50/50) | CG110 | | |
| | polyglucoside | | | |
| | (2) in a | | | |
| | buffered 60% | | | |
| | aqueous | | | |
| | solution | | | |
| | Demineralized | | 28 | g |
| | water | | | |
| | Pure absolute | Prolabo | 20 | g |
| | ethanol | | | |
| | Pure benzyl | Fluka | 4 | g |
| | alcohol | | | |
| | Polyethylene | | 6 | g |
| | glycol (8 EO) | | | |
| | 400 | | | |
| | Demineralized | | qs 100 | g |
| | water | | | |
| Preparation | Ammonium | Prolabo | 54 | g |
| of buffer pH | chloride | | | |
| 9.5 | (NH₄Cl) | | | |

| | |
|---|---|
| Aqueous | qs pH 9.5 |
| ammonia in | (approx. |
| solution at | 20 ml) |
| 20% | |
| Demineralized | qs 1000 ml |
| water | in a |
| | volumetric |
| | flask |

ii) Study of penetration on diffusion cells:
   application temperature: 32° C.
   dose applied: 250 μl cm⁻²
   application time: 4 h
   recipient liquid: PBS, 0.25% Tween 80 (with 2.5 mM Na-vitaminized).
iii) The recipient liquid is collected and then analysed in duplicate by HPLC. The concentration is determined using a standard range prepared on the same day from a stock solution at 0.5 mM in PBS, 0.25% Tween 80³.
iv) The degree of penetration is calculated by relating the concentration measured to that of the dose applied, taking into account the volumes, i.e.: 70 μl of formulation and 600 μl of recipient liquid.

HPLC Method and Chromatogram

The HPLC system used is an Alliance 2695 system and a Waters PAD 996 detector, used according to the manufacturer's specifications.

For the analysis, aliquots of the standard-range solutions and of the samples to be assayed were taken and dispensed into vials equipped with an insert. The dye sample quantification was carried out using the Millenium v.3.2 acquisition and processing system.

Condition A (i.e. conventional Episkin® condition) uses the Episkin® differentiation medium as culture medium, which is optimal for the differentiation of the model. The batch B condition (i.e. negative control) therefore uses a culture medium with 30% less of this differentiation medium. Thus, the barrier function exhibited by condition B was therefore found to be lower than that of condition A.

The following table gives the degrees of penetration of the reference compound for conditions A, B and C of the Episkin® batches used (condition at D14).

| | | | 03-epis- |
|---|---|---|---|
| | | | 4/07/2003 |
| Condition A | Batch A conventional medium | mean | 7.96 |
| | | SD | 0.52 |
| | | CV | 6.52 |
| Condition B | Batch B (+30% non- | mean | 9.78 |
| | stimulated medium) | SD | 2.21 |
| | | CV | 22.59 |
| Condition C | Batch C (+30% medium | mean | 7.15 |
| | stimulated with probiotic | SD | 0.25 |
| | L. paracasei + | CV | 3.45 |
| | Bifidobacterium lactis NCC | | |
| | 2818) | | |

The results show a significant improvement in the barrier function compared with the negative control.

Only condition C results in a significant improvement in the barrier function compared with the negative control. The use of conditioned medium stimulated with the probiotics makes it possible to obtain a barrier function equivalent to that obtained under standard conditions.

### Conclusions

Thus, it is demonstrated that the condition supplemented with 30% of conditioned media stimulated with probiotics (condition C) results in a significant improvement in the barrier function.

### EXAMPLE 3

### Restoration of the Barrier Function

The aim of this study was to evaluate the effect of probiotics on the restoration of the barrier function (BF) of fresh excised skin isolated in a diffusion cell.

The influence of the conditioned media derived from cocultures stimulated with the probiotic L. paracasei, obtained as described in Example 1, introduced into the recipient liquid, was studied. This makes it possible to evaluate the effect on restoration of the barrier function, impaired by sodium lauryl sulphate (SLS). For this, human skin fragments recovered immediately post-operatively were placed in a Franz® cell system in order to measure the insensible water loss (IWL) over time.

The evaluation was carried out on samples of fresh abdominoplasty. At D10, the skin fragments were placed in a Franz® cell at 32° C. The skin fragments in a Franz® cell have a surface area of 10.18 cm².

The culture medium suitable for maintaining under survival conditions was added to the recipient compartment of the Franz cells (5.5 ml), supplemented with the conditioned culture media in the proportion of 30% (this medium is the one described by Boisnic S, Branchet-Gumila M C, Merial-Kieny C, Nocera T; Skin Pharmacol Physiol 2005: 18: 201-208).

The conditions to be tested are the following:

CONTROL CONDITION 2 on which there will be a treatment with SLS (“singly”): non-stimulated conditioned medium (called medium 2), corresponding to a simple conditioned culture medium brought into contact with the reconstructed model (Caco2/PBMC).

CONDITION 3 (in “duplicate”): stimulated conditioned medium (called medium 3) corresponding to a conditioned culture medium derived from the stimulation, with the probiotic L. paracasei, of an in vitro model representative of the intestinal system (Caco2/PBMC) (stimulation by L. paracasei at 10⁸ cfu/ml brought into contact with the Caco2/PBMC reconstructed model for 16 h).

CONDITION 4 on which there will be a treatment with SLS (in “duplicate”): stimulated conditioned medium (called medium 3), corresponding to a conditioned culture medium derived from the stimulation, with the probiotic L. paracasei, of an in vitro model representative of the intestinal system (Caco2/PBMC) (stimulation with L. paracasei at 10⁸ cfu/ml brought into contact with the Caco2/PBMC reconstructed model for 16 h of stimulation).

2 donors are analysed after impairment of the barrier function according to the following protocol:

At D0, the skin is prepared and mounted on Franz cells in the presence of the culture medium.

Said cells are then left for 1 h in order for them to re-equilibrate, in particular with respect to the pH and water exchanges, before a first measurement of IWL. 3 h after the mounting, the conditioned media are added as pretreatment overnight in the Franz cells in the culture medium.

At D1

After contact overnight with the conditioned media, a first measurement of the IWL is carried out in the morning at around 8.30 am.

The SLS is then applied under occlusion at 10%: in order to obtain a significant but reversible impairment of the barrier function of our Franz-cell-mounted explant, 20 μl per cm² must be applied for 24 h under partial occlusion (by virtue of a paraffin sheet stretched over the donor compartment).

At D2

The surplus SLS is cleaned off with 600 μl of milliQ water (3 washes and then drying of the skin by dabbing with a cotton-wool bud).

The medium in the Franz cells is changed.

The IWL is measured 1 hour later, and then every 2 h (4 times a day).

At D3

At 8.30 am, the IWL is measured and then the medium is changed.

In addition, the IWL is measured every 2 hours (4 times a day).

At D4

The IWL is measured at 8.30 am and the manipulation is stopped.

The IWL was measured in duplicate by means of an analysed Franz cell. The probe was placed at the surface of the skin samples.

Results

1st skin

| D0 at T1h | 5.2 ± 1.7 | | |
|---|---|---|---|
| D0 at T4h | Condition 2 = | Condition 3 = | Condition 4 = |
| | medium 2 added | medium 2 or 3 | medium 3 added |
| | | added | |
| D1 at T20h | ±0.8 | 4.45 ± 1.8 | |
| after | | | |
| mounting of | | | |
| Franz cells | | | |
| D1 + SLS | SLS applied | | SLS applied |
| | for 3 h | | for 3 h |
| D1 at T1h* | 20.7 ± 0 | 5.5 ± 2.2 | 10.5 ± 1 |
| D1 at T3h* | 16.25 ± 1.25 | 5.4 ± 1.9 | 9.5 ± 0.8 |
| D2 at T19h* | 7.65 ± 0.65 | 4.7 ± 3.1 | 5.55 ± 1 |
| D2 at T21h* | 7.95 ± 0.15 | 5.6 ± 2.6 | 7.95 ± 0.2 |
| D2 at T23h* | 9.5 ± 1.2 | 5.1 ± 2.95 | 8 ± 0.2 |
| D2 at T26h* | 7.6 ± 0.4 | 5.07 ± 2.8 | 6.65 ± 0.1 |
| D2 at T29h* | 7.3 ± 0.1 | 4.8 ± 1.7 | 9.25 ± 0.75 |
| D3 at T45h* | 7.55 ± 0.45 | 5.3 ± 2.3 | 6.45 ± 1.5 |

*time counted from after treatment of the cells concerned with SLS

The conditioned medium stimulated with L. paracasei at 10⁸ cfu according to the invention limits the impairment of the barrier function impaired with SLS and allows a more rapid restoration of this function, identical to the normal level (return to the values of IWL found on the fragments on which no SLS treatment was applied).

2nd skin:

| D0 at T1h | 6 ± 1.7 | | |
|---|---|---|---|
| D0 at T4h | Condition 2 = | Condition 3 = | Condition 4 = |
| | medium 2 added | medium 3 added | medium 3 added |
| D1 at T20h | ±1 | 5 ± 0.9 | |
| after | | | |
| mounting of | | | |
| Franz cells | | | |
| D1 + SLS | SLS applied | | SLS applied |
| | for 3 h | | for 3 h |
| D1 at T1h* | 22.85 ± 1.25 | 3 ± 0.3 | 12.65 ± 0.5 |
| D2 at T19h* | 11.9 ± 2 | 3.35 ± 1 | 8 ± 1.6 |
| D2 at T21h* | 11.75 ± 0.45 | 3.4 ± 0.46 | 5.35 ± 0.7 |
| D2 at T23h* | 12.5 ± 0.5 | 3.25 ± 0.4 | 5.8 ± 0.2 |
| D2 at T26h* | 14 ± 0.2 | 3.4 ± 0.5 | 9.9 ± 2.1 |
| D3 at T45h* | 8 ± 0.1 | 3.7 ± 0.3 | 4 ± 0.6 |

*time counted from after treatment of the cells concerned with SLS

As in the previous case, the treatment with conditioned medium according to invention limits the impairment of the barrier function impaired with SLS and allows a rapid restoration of this barrier function.

### EXAMPLE 4

### Topical Compositions to be Applied to the Skin or the Scalp:

(The ingredients are indicated under their CTFA names)

Composition for preparing the skin for sunlight

| | |
|---|---|
| Stimulated conditioned medium* | 2.5% |
| Preserving agents | 1.35% |
| Sodium citrate | 0.035% |
| PEG-40 | 1.25% |
| Pentaerythrityl tetraethylhexanoate | 4% |
| Glycerol | 7% |
| Sorbitan tristearate | 0.3% |
| Prunus armeniaca Kermel oil | 2% |
| Cetyl alcohol | 0.7% |
| Propylene glycol | 2% |
| Triethanolamine | 0.4% |
| Cyclohexasiloxane | 2% |
| Carbomer | 0.75% |
| Tocopherol | 1% |
| Silica | 2% |
| Ascorbyl glucoside | 0.1% |
| Polycaprolactone-beta carotene | 5% |
| Water qs | 100% |

*obtained according to Example 1

Composition containing sunscreens

| | |
|---|---|
| Stimulated conditioned medium** | 3.5% |
| Mixture of cetylstearyl alcohol and of | 7.0% |
| oxyethylenated cetylstearyl alcohol | |
| (33 EO) 80/20 | |
| Mixture of glyceryl monostearate | 2.0% |
| and distearate | |
| Cetyl alcohol | 1.5% |
| Polydimethylsiloxane | 1.5% |
| Liquid petroleum jelly | 15.0% |
| Butylmethoxydibenzoylmethane | 3.0% |
| Octocrylene | 7.0% |
| Glycerol | 20.0% |
| Demineralized water | qs 100% |

**obtained according to Example 1, but with L. johnsonii (CNCM No. 1225) as probiotic

Composition for treating hair loss: hair lotion

| | |
|---|---|
| Stimulated conditioned medium*** | 1% |
| Propylene glycol | 23% |
| Ethanol | 55% |
| Aminexil | 1.5% |
| Water | qs 100% |

***obtained according to Example 1, but with B. longum (CNCM No. 2170) as probiotic

Composition containing antioxidants for protecting the
skin against damage caused by pollution, cigarette
smoke

| | |
|---|---|
| Stimulated conditioned medium**** | 2.0% |
| Hydroxypropylcellulose (Klucel H sold by the | 1.0% |
| company Hercules) | |
| Antioxidant-vitamin E | 2.0% |
| Isopropanol | 40.0% |
| Preserving agent | 0.30% |
| Water | qs 100% |

****obtained according to Example 1, using a mixture of L. paracasei and B. longum

Body lotion for dry skin

| | |
|---|---|
| Mineral oil | 8.0% |
| Isopropyl palmitate | 5.0% |
| Polyglyceryl-3 diisostearate | 4.0% |
| Octyldodecanol | 4.0% |
| Carbomer | 0.3% |
| Stimulated conditioned medium* | 2.0% |
| Sodium cocoyl glutamate | 2.0% |
| Preserving agent | 0.5% |
| Fragrance | 0.5% |
| Water | qs 100% |

*obtained according to Example 1

Anti-dandruff shampoo

| | |
|---|---|
| Sodium lauryl sulphate | 7.0% |
| Cocamidopropylbetaine | 2.0% |
| Sodium lauryl sulphosuccinate | 2.0% |
| Conditioned medium extract** | 4.0% |
| Sodium chloride | 1.0% |
| Preserving agents | 0.5% |
| Fragrance | 0.5% |
| Water | qs 100% |

**lyophilized extract obtained according to Example 1 but with B. lactis (NCC2818) as probiotic

Cream

| | |
|---|---|
| Arachidyl behenyl alcohol/arachidyl glucoside | 3.0% |
| Isohexadecane | 7.0% |
| Sweet almond oil | 3.0% |
| Shea butter | 2.0% |
| Glycerol | 5.0% |
| Stimulated conditioned medium* | 3.0% |
| BHT | 0.05% |
| Methyl POB | 0.1% |
| Propyl POB | 0.05% |
| Water | qs 100% |

*obtained according to Example 1

Cream

| | |
|---|---|
| Conditioned medium extract***** | 1.5% |
| Glyceryl stearate and PEG 100 stearate | 5.0% |
| Isohexadecane | 8.0% |
| Shea butter | 5.0% |
| Glycerol | 3.0% |
| Carbopol 981 0.2% | 0.2% |
| Lubragel | 5.0% |
| Phenoxyethanol | 1.0% |
| Sodium hydroxide | qs pH 6 |
| BHT | 0.05% |
| Dc 1503 | 1.0% |
| Water | qs 100% |

*****lyophilized extract obtained according to Example 1 but with B. bifidum as probiotic.

The above written description of the invention provides a manner and process of making and using it such that any person skilled in this art is enabled to make and use the same, this enablement being provided in particular for the subject matter of the appended claims, which make up a part of the original description and including a composition, for example a cosmetic or dermatological composition, comprising a conditioned cell culture medium or an extract thereof, said medium being able to be obtained, or being obtained, by contact with at least one culture of digestive tract cells and at least probiotic microorganisms, or by contact with at least one culture of digestive tract cells and at least one probiotic microorganism.

As used herein, the phrases “selected from the group consisting of,” “chosen from,” and the like include mixtures of the specified materials. Terms such as “contain(s)” and the like as used herein are open terms meaning ‘including at least’ unless otherwise specifically noted. Phrases such as “mention may be made,” etc. preface examples of materials that can be used and do not limit the invention to the specific materials, etc., listed.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. In this regard, certain embodiments within the invention may not show every benefit of the invention, considered broadly.

1. A composition comprising a conditioned cell culture medium or an extract thereof, said medium being obtainable by contact with at least one culture of digestive tract cells and at least one probiotic microorganism.

2. The composition according to claim 1, wherein said medium is obtained by contact with at least one culture of digestive tract cells, at least one probiotic microorganism, and peripheral blood cells.

3. The composition according to claim 2, wherein the peripheral blood cells are leukocytes.

4. The composition according to claim 1, wherein the culture of digestive tract cells is a three-dimensional culture.

5. The composition according to claim 2, wherein the culture of digestive tract cells is a three-dimensional culture.

6. The composition according to claim 3, wherein the culture of digestive tract cells is a three-dimensional culture.

7. The composition according to claim 1, wherein the digestive tract cells are intestinal epithelial cells.

8. The composition according to claim 1, wherein the at least one probiotic microorganism is chosen from the following group: Saccharomyces, Yarrowia, Kluyveromyces, Torulaspora, Schizosaccharomyces pombe, Debaromyces, Candida, Pichia, Aspergillus, Penicillium, Bifidobacterium, Bacteroides, Fusobacterium, Melissococcus, Propionibacterium, Enterococcus, Lactococcus, Staphylococcus, Peptostrepococcus, Bacillus, Pediococcus, Micrococcus, Leuconostoc, Weissella, Aerococcus, Oenococcus, Lactobacillus and mixtures thereof.

9. The composition according to claim 1, wherein the at least one probiotic microorganism is chosen from lactic acid bacteria, bifidobacteria, Saccharomyces yeasts, and mixtures thereof.

10. The composition according to claim 1, wherein the at least one probiotic microorganism is chosen from the Lactobacillus and Bifidobacterium species.

11. A process for improving skin homeostasis, comprising contacting the skin with a conditioned cell culture medium or an extract thereof, said medium being obtained by contact with at least one culture of digestive tract cells and at least one probiotic microorganism.

12. The process of claim 11, wherein said medium is obtained by contact with at least one culture of digestive tract cells, at least one probiotic microorganism, and leukocytes.

13. The process of claim 11, wherein said process is a process for improving the barrier function of the skin or of the scalp, and said conditioned cell culture medium or an extract thereof is applied to the skin and/or scalp of a person in need thereof.

14. The process of claim 11, wherein said process is a process for improving the moisturization of the skin or of the scalp, and said conditioned cell culture medium or an extract thereof is applied to the skin and/or scalp of a person in need thereof.

15. The process of claim 11, wherein said process is a process for delaying, reducing and/or preventing the signs of skin ageing, and said conditioned cell culture medium or an extract thereof is applied to the skin of a person in need thereof.

16. The process of claim 11, wherein said process is a process for improving skin homeostasis and/or the barrier function of the skin or of the scalp, and said conditioned cell culture medium or an extract thereof is applied to the skin and/or scalp of a person in need thereof.

17. The process of claim 11, wherein said process is a process for the treatment of sensitive skin, and said conditioned cell culture medium or an extract thereof is applied to the skin of a person in need thereof.

18. The process of claim 11, wherein said process is a process for the treatment of signs of skin stress, and said conditioned cell culture medium or an extract thereof is applied to the skin of a person in need thereof.

19. A method for the preparation of an in vitro skin equivalent, comprising the use of a conditioned cell culture medium or an extract thereof, said medium being obtained by contact with at least one culture of digestive tract cells and at least one probiotic microorganism.

20. A composition according to claim 1 comprising a conditioned cell culture medium or an extract thereof, said medium being obtained by contact with at least one culture of digestive tract cells and at least one probiotic microorganism.

21. A method for preparing the composition according to claim 1, comprising:
a) culturing cells derived from an intestinal epithelium on a porous support in a first nutritive medium for a period of time sufficient to obtain their differentiation;
b) preparing a second cell culture medium in a chamber covered with a carpet of leukocytes;
c) recovering the culture of differentiated cells on the porous support obtained at the end of a), and transferring to the second culture medium obtained at the end of b);
d) bringing said culture of differentiated cells derived from the intestinal epithelium in the second culture medium into contact with a culture of microorganisms comprising at least a probiotic of the Lactobacillus species, for a period of time sufficient for there to be an interaction between the cells;
e) eliminating the cell cultures and recovering the second cell culture medium, freed of the leukocytes, so as to obtain a conditioned medium.
